(19) United States
(12) Patent Application Publication
(10) Pub. No.: US 2010/0029757 A1
(43) Pub. Date: Feb. 4, 2010

(54) USE OF XANTHOHUMOL AND/OR ISOXANTHOHUMOL AS AN AGENT FOR PREVENTING AND/OR COMBATING LIVER DISEASES

(75) Inventor: Claus Hellerbrand, Regensburg (DE)

Correspondence Address: HAMRE, SCHUMANN, MUELLER & LARSON, P.C., P.O. BOX 2902, MINNEAPOLIS, MN, 55402-0902, US

(73) Assignee: JOH. BARTH & SOHN GMBH & CO. KG, Nurnberg (DE)

(21) Appl. No.: 12/520,756

(22) PCT Filed: Dec. 21, 2007
(86) PCT No.: PCT/EP07/11358

§ 371 Date: Sep. 21, 2009

(30) Foreign Application Priority Data
Dec. 22, 2006 (DE) 10 2006 062 264.2

(51) Int. Cl. A61K 31/352; C07C 49/84; C07D 311/34; A61P 3/00; A61P 3/10; A61K 31/12; A61P 1/16

(52) U.S. Cl. 514/456; 568/334; 549/403; 514/685

(57) ABSTRACT

The present invention relates to the use of xanthohumol with formula (I) as an agent for the production of a preparations for preventing and/or combating liver diseases.

The present invention relates to the use of xanthohumol or isoxanthohumol as an active substance for the prevention and/or control of liver diseases.

### BACKGROUND

Xanthohumol is a prenylflavonoid which occurs in hops. Various studies have demonstrated the biological effects of xanthohumol.

For example, the anticarcinogenic effect of xanthohumol is described in EP 1 543 834 A1.

It is known from EP 0 679 393 B1 that xanthohumol has a strong inhibitory effect on bone absorption, and therefore may be used as an agent for the treatment of osteoporosis.

DE 103 08 864 A1 describes a novel brewing method for producing a beer which due to a special brewing process contains an elevated concentration of xanthohumol and therefore has increased health-promoting effects.

### OBJECT OF THE INVENTION

The object of the present invention is to find further health-promoting applications for xanthohumol and isoxanthohumol.

This object is achieved by the use of xanthohumol as an active substance for producing a preparation for the prevention and/or control of liver diseases.

The above object is further achieved by the use of isoxanthohumol as an active substance for producing a preparation for the prevention and/or control of liver diseases.

The claimed use has the advantage that by use of a natural active substance, liver diseases may be prevented and, through treatment, effectively eliminated or controlled. Xanthohumol or isoxanthohumol have no side effects. This allows effective prophylactic protection from chronic liver diseases over a long time period, in particular when taken regularly.

Xanthohumol and isoxanthohumol are particularly well suited for prevention or treatment of acute cirrhosis of the liver or hepatic fibrosis. Surprisingly, studies have shown that xanthohumol inhibits metabolic mechanisms which are very important for liver damage mediated by adiposis (overweight) and diabetes. Adiposis and diabetes are responsible for the majority of cases of cirrhosis of the liver, and the trend is increasing. Collectively, chronic liver diseases have come to represent a significant economic problem. By continuous administration of xanthohumol or isoxanthohumol it is possible to provide effective prophylactic protection, without side effects, for the entire population.

Studies have further shown that xanthohumol or isoxanthohumol also have antiviral properties, and exhibit very good activity against hepatitis, in particular hepatitis B and hepatitis C. Hepatitis B or hepatitis C is the most common causative factor in chronic liver disease. Epidemiological studies in Germany have shown that approximately 2% of the population is infected with chronic hepatitis B or hepatitis C. This is also a problem of key social significance. By prophylactic administration of xanthohumol or isoxanthohumol it is possible on the one hand to effectively reduce the number of hepatitis cases, i.e., cases of hepatitis B and C, and on the other hand to favorably influence the course of an existing case of hepatitis.

There are currently no proven therapeutic administration forms for the treatment of hepatic fibrosis. Fibrosis can be inhibited or halted only by elimination of the harmful root cause, i.e., in the case of a hepatitis virus infection, for example, by elimination of the hepatitis virus. However, elimination of the root cause is successful in only a percentage of patients with chronic liver disease, and as a rule is not possible for patients with genetic liver disease. In the case of hepatitis virus infections, it has been necessary thus far to use medicaments having strong side effects. Even when such medicaments are used, elimination of the virus is achieved in only a percentage of patients. Fortunately, the use of xanthohumol or isoxanthohumol may provide a remedy.

Lastly, xanthohumol and isoxanthohumol also have anticarcinogenic effects. For liver cancer or hepatocelluar carcinoma (HCC), besides surgery there is currently no proven therapy which would improve the survival rate of patients. At the present time, surgical removal is successful only in a very small percentage of HCC patients, since by the time that diagnosis is made the HCC has usually become too large or has formed metastases. It has been shown that xanthohumol may be used in the treatment of liver cancer.

Furthermore, xanthohumol or isoxanthohumol may be used as a preventative specifically in persons with a high risk profile (genetic risk, persons with adiposis, diabetics).

With regard to administration, the invention provides for use by supplying xanthohumol or isoxanthohumol as an active ingredient of a pharmaceutical composition together with a pharmaceutically acceptable carrier such as mannite, sucrose, lactose, glucose, fructose, maltose, etc.

Xanthohumol or isoxanthohumol is particularly suitable when added as an active substance to a food, or mixed with a beverage.

According to one practical embodiment of the use according to the invention, xanthohumol or isoxanthohumol as an active substance is added in particular to reduce or suppress the activity of free oxygen radicals specifically in the liver. It has been found that when liver damage is present in any form, i.e., as the result of inflammation (for example, from viruses, excessive alcohol consumption, obesity, and/or diabetes or radiation exposure), free oxygen radicals are formed which may play a key role in the development of liver inflammation, hepatic fibrosis or cirrhosis of the liver, and liver cancer. Xanthohumol or isoxanthohumol inhibits the formation of free oxygen radicals or interferes with their activity. This results in the advantage that all three of the above-referenced damage mechanisms for the liver may be effectively influenced in equal measure by xanthohumol or isoxanthohumol.

In particular, it has been found that adding xanthohumol or isoxanthohumol is particularly suited for crucially influencing the NF-kappa B factor, i.e., in particular for reducing or suppressing the activity of the NF-kappa B factor. NF-kappa B is a signal mediator in the cell, and participates in the modulation of numerous cell functions. It has been found that the NF-kappa B factor plays a major role specifically in the three above-referenced damage mechanisms for the liver. NF-kappa B also plays an important role in the development and progression of NASH.

Furthermore, it has surprisingly been found that xanthohumol or isoxanthohumol may be administered in comparatively high dosages. According to the findings, no harmful effect from xanthohumol or isoxanthohumol occurs in any of the cells, even at high dosages, thus resulting in selective activity.

It has been found that with increasing dosages of xanthohumol or isoxanthohumol, for example beginning at a lower limit of 5 μM, a continuous increase in the positive effect can be observed, in particular up to a maximum limit of 100 μM. This results in the advantage that, depending on the intended use of the treatment agent (food with a proportion of xanthohumol or isoxanthohumol for daily intake as a preventative, or as a medicament for treatment), preparations having different dosages may be marketed for specific purposes.

For example, in a chronic infection several activity mechanisms may be present at the same time, so that the preparation according to the invention may be used to appropriately control the three activity mechanisms for liver inflammation, cirrhosis of the liver or hepatic fibrosis, and development of liver cancer as well as progression of liver cancer, all at the same time.

It is practical to use the active substance, i.e., the xanthohumol or isoxanthohumol or a metabolite thereof or a precursor thereof, in an administration form (application and/or dosage) which results in active substance concentrations of ≧5 μM, in particular ≧10 μM, in particular ≧20 μM, in particular ≧30 μM, in particular ≧40 μM, in particular ≧50 μM, in the liver.

The active substance is preferably used in an administration form which results in a maximum active substance concentration of 100 μM in the liver.

Depending on the application, the particular active substance should be used in an administration form in such a way that the following ranges of active substance concentrations result in the liver: 1 to 100 μM, preferably 1-25 μM, preferably 1-10 μM, or 5-100 μM, preferably 10-50 μM, preferably 10-25 μM. The applicable ranges may be selected depending on the application. In particular, comparatively low doses are sufficient for the treatment of fibrosis, whereas increased doses are practical for treatment of liver cancer.

When xanthohumol or isoxanthohumol is administered in food or as a tablet, for example, due to absorption by the intestine relatively high xanthohumol or isoxanthohumol levels may result, but these are rapidly diluted after passage through the liver; i.e., no other organ has anywhere near such a high xanthohumol level.

With regard to recovery of xanthohumol from hops plants, reference is made to the entire disclosures of EP 0 679 393 B1 and EP 1 543 834 A1.

Instead of xanthohumol or isoxanthohumol, according to the present invention a metabolite thereof may also be used, in particular a metabolite which is produced in the liver by the P450 enzyme complex. Such metabolites are primarily xanthohumol glucoronides or sulfates, and methylated forms of xanthohumol or naringenins, in particular 8-prenylnaringenin. Naringenin, in particular 8-prenylnaringenin, is the final metabolite of xanthohumol.

Likewise, instead of xanthohumol or isoxanthohumol a precursor thereof may be used which regenerates to form xanthohumol under chemical and/or physiological conditions.

All of the uses of xanthohumol and isoxanthohumol described in the present patent application for the treatment of liver diseases therefore also apply for the above-described metabolites and precursors.

According to the present invention, as active substance a composition may be used in which the xanthohumol and/or isoxanthohumol are present not in the pure form, but rather in the form of a hops extraction product. It has been found that in addition to xanthohumol or isoxanthohumol, carrier constituents are present as the result of the production process which are able to further assist in absorption of the active substance into the organism and thereby help boost efficacy.

The dosage for administration of the active substance relative to the respective (pure) fraction of active substance is advantageously greater than 0.01 mg/kg body weight/day, preferably greater than 0.1 mg/kg body weight/day, preferably greater than 1 mg/kg body weight/day, preferably greater than 10 mg/kg body weight/day, preferably greater than 50 mg/kg body weight/day, preferably greater than 100 mg/kg body weight/day, whereby the body weight refers to the body weight of a person.

The dosage for administration relative to the respective (pure) fraction of active substance is advantageously less than 161 mg/kg body weight/day, preferably less than 50 mg/kg body weight/day, preferably less than 10 mg/kg body weight/day, preferably less than 1 mg/kg body weight/day, preferably less than 0.1 mg/kg body weight/day, whereby the body weight refers to the body weight of a person.

The dosage for administration relative to the respective (pure) fraction of active substance is advantageously in a range of 0.01 to 161 mg/kg body weight/day, preferably 0.05 to 120 mg/kg body weight/day, preferably 0.1 to 100 mg/kg body weight/day, preferably 0.5 to 80 mg/kg body weight/day, preferably 1 to 80 mg/kg body weight/day, preferably 5 to 80 mg/kg body weight/day, preferably 10 to 80 mg/kg body weight/day, whereby the body weight refers to the body weight of a person.

The proportions of xanthohumol or isoxanthohumol advantageously are in a range of 0.1 wt-%-99 wt-%, preferably 5 wt-%-99 wt-%, preferably 10 wt-%-99 wt-%, preferably 20 wt-%-99 wt-%, preferably 30 wt-%-99 wt-%, preferably 40 wt-%-99 wt-%, preferably 50 wt-%-99 wt-%, preferably 60 wt-%-99 wt-%, preferably 70 wt-%-99 wt-%.

If further constituents, in particular natural constituents resulting from the recovery of xanthohumol from hops, are present in addition to the xanthohumol as active substance, this may even increase the efficacy, since these constituents result in improved absorption of the active substance in the organism.

Alternatively, the xanthohumol or isoxanthohumol may also be used in pure form.

In addition, according to the present invention it is also possible to use xanthohumol or isoxanthohumol in synthesized form.

According to a further embodiment, the xanthohumol, isoxanthohumol, a metabolite thereof, and/or a precursor thereof are used in combination with at least one additional active substance. This active substance may preferably be one which positively influences the tolerability and/or absorption in the body, and/or the efficacy and/or stability and/or handling characteristics, of the active substance to be administered.

The xanthohumol, isoxanthohumol, a metabolite thereof, and/or a precursor thereof may be used in combination with or on the basis of a salt, in particular an alkali or alkaline earth salt.

The agent to be administered may in particular be used in the form of a liquid, suspension, or emulsion, in the form of nanoparticles, or as a powder or gel. The administration may be carried out as an independent medicament, or also as an additive to a liquid or solid food, depending on whether therapy or prophylaxis is desired.

The active substance may be administered using solvents, carrier substances, or additives such as starches, dextrin, in particular cyclodextrin or maltodextrin, proteins, methyl cellulose, carbomethoxycellulose, or xanthan gum which are suitable for pharmaceuticals, nutrients, or foods.

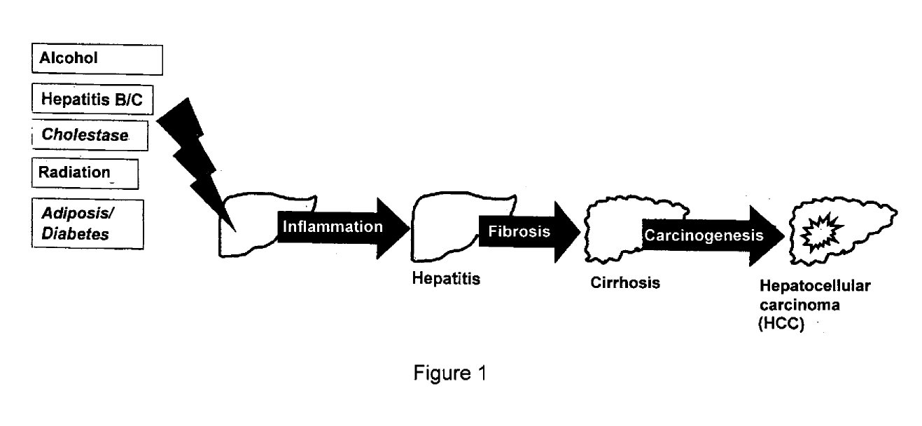

The studies according to the following FIGS. 1-9 were carried out using xanthohumol in pure form (>98%).

### EXAMPLE 1

A composition of a medicament is provided below as an example.

Powdered mixture for direct pressing

| | | |
|---|---|---|
| Xanthohumol (pure substance) | 5 | g |
| Microcrystalline cellulose | 10 | wt-% |
| Sodium carboxymethyl starch | 3 | wt-% |
| Highly dispersed silica | 1 | wt-% |
| Magnesium stearate | 1 | wt-% |
| Tablettose (lactose monohydrate) | to make 100 | wt-% |

### EXAMPLE 2

A composition of a food with added xanthohumol as active substance is provided below as an example.
Xanthohumol (pure substance in powdered form) 500 mg per 200 mL
milk product (creamy, for example yogurt)

Due to the ease of admixture into a creamy food, the above composition for a food allows optimal administration of the required quantity of xanthohumol.

FIG. 1 shows the therapeutic objectives for the use of xanthohumol. The illustration represents the chain of activity mechanisms, starting from a liver disease resulting from alcohol, viruses, radiation, adiposis, and/or diabetes, for example, all the way to liver cancer. The use of a preparation containing xanthohumol and/or isoxanthohumol advantageously interferes with all stages of the activity chain according to FIG. 1. However, xanthohumol or isoxanthohumol may also be used successfully in a targeted manner in the treatment of individual stages of the activity sites.

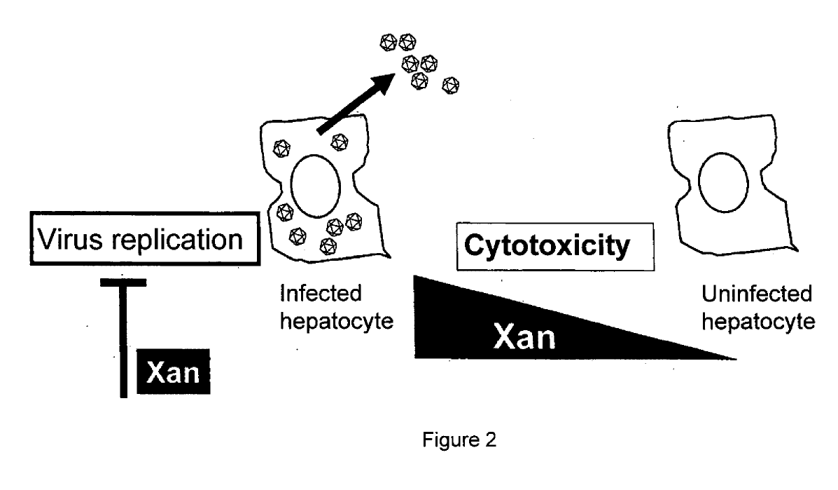

FIG. 2 shows a schematic illustration of the efficacy of xanthohumol and/or isoxanthohumol for viral damage to the liver, in particular as the result of hepatitis B and C. It has been found that the above-referenced active substances advantageously not only inhibit replication of the virus, but also ensure selective destruction of the body's own liver cells already affected by the virus while leaving healthy liver cells undamaged. Thus, use of the invention allows a targeted therapy for reduction or elimination of liver cells infected with the virus.

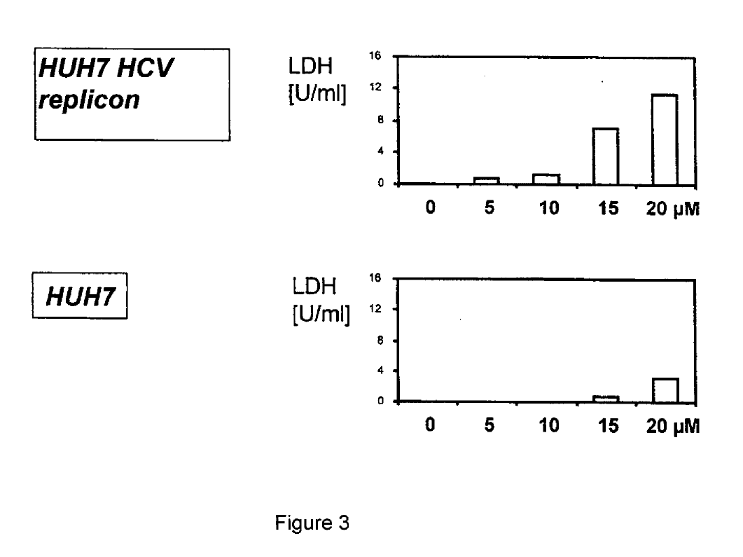

On the basis of comparative diagrams, FIG. 3 shows the selective efficacy for the use of xanthohumol or isoxanthohumol for liver cells infected with hepatitis C, compared to liver cells not infected with hepatitis C.

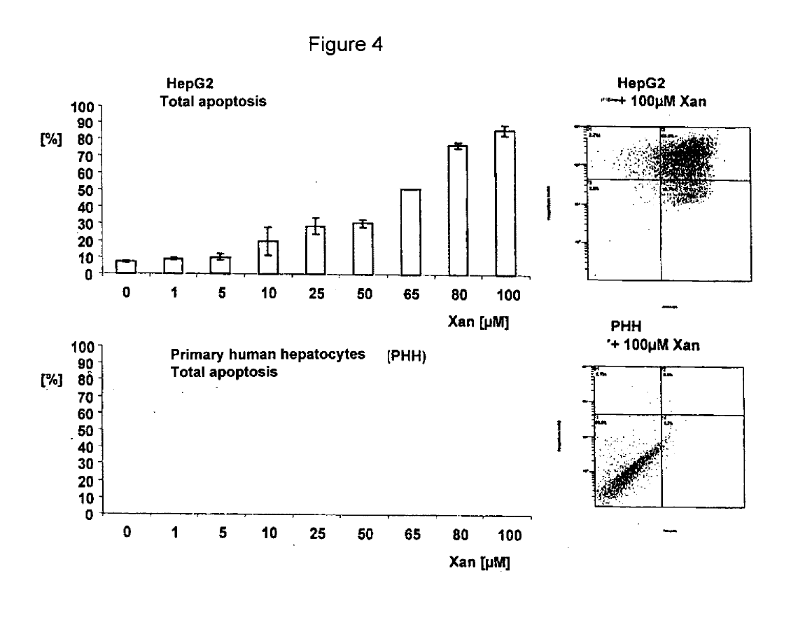

FIG. 4 shows a graphical illustration of the efficacy of the use of xanthohumol with regard to apoptosis (programmed cell death) of liver cancer cells (HepG2) compared to healthy liver cells (primary human hepatocytes).

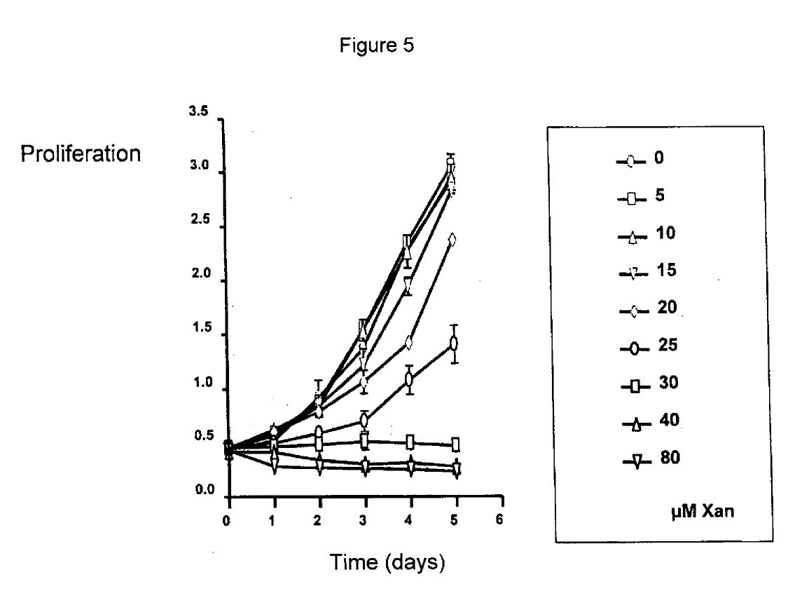

FIG. 5 shows a comparison of the growth of liver cancer cells (HepG2) over time as a function of the dosage of xanthohumol. As clearly shown in the illustration, the growth of the cancer cells is progressively inhibited with increasing concentrations of xanthohumol.

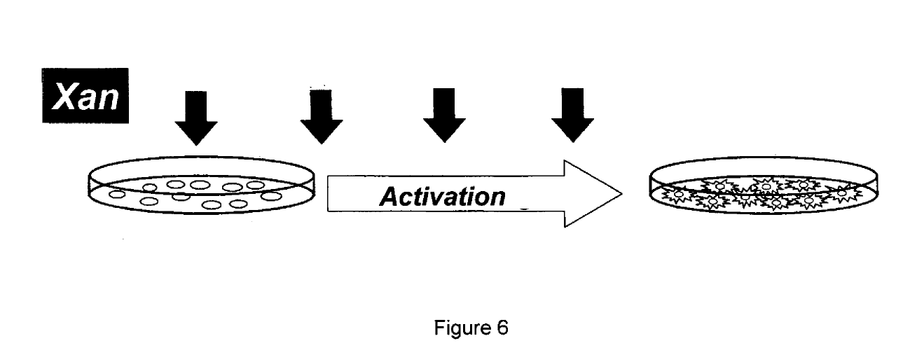

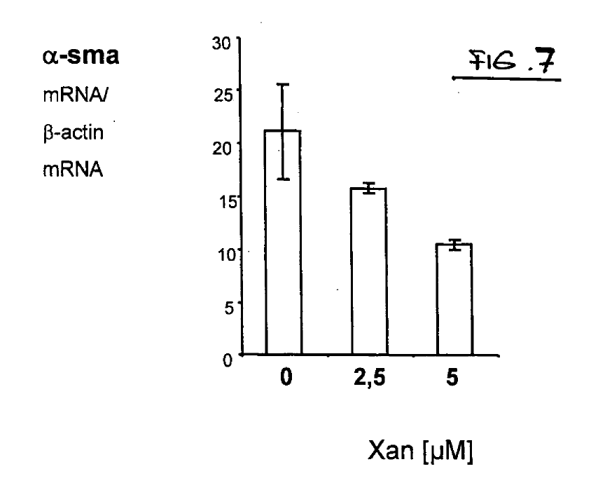

FIGS. 6 and 7 illustrate the effect of addition of xanthohumol for the prevention of transformation of the body's own liver cells to hepatic stellate cells, which are responsible for scarring of the liver in cirrhosis of the liver.

As shown in FIG. 7, the formation of scar tissue is increasingly suppressed as the dosage of xanthohumol increases.

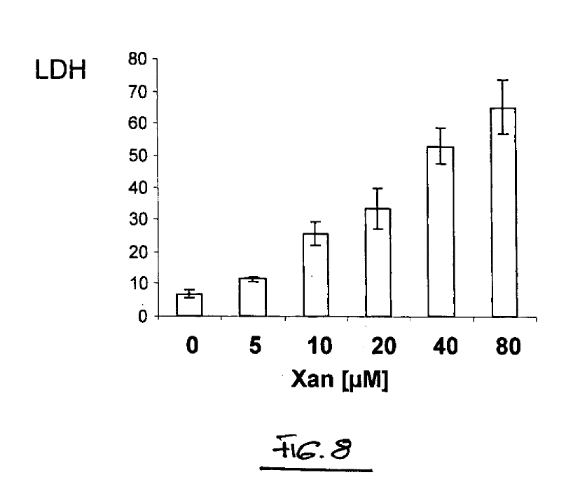

FIG. 8 shows an illustration of the effect of increasing dosages of xanthohumol on the activated hepatic stellate cells already present. From the illustration according to FIG. 6 [sic; 8] it is seen that an increased effect of destruction (LDH) of activated hepatic stellate cells results from an increasing dosage of xanthohumol.

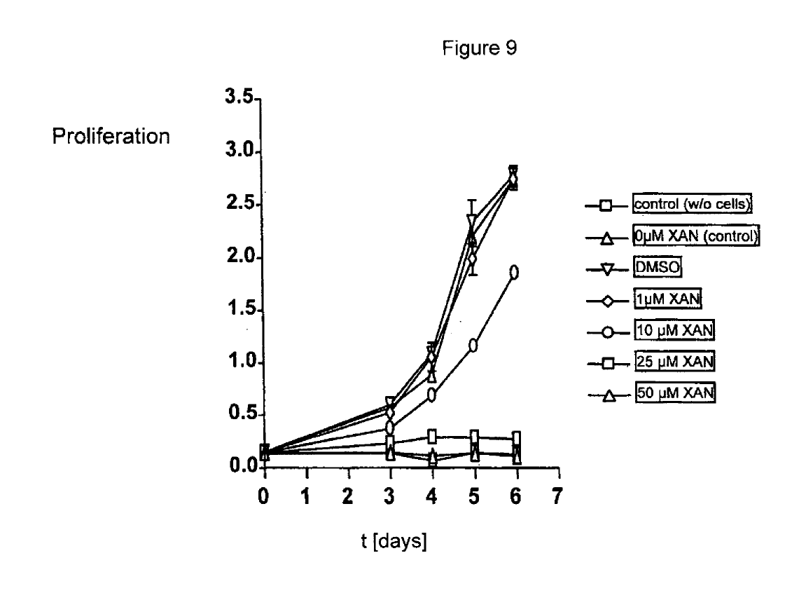

FIG. 9 shows the influence of the dosage of xanthohumol on the growth of the activated hepatic stellate cells.

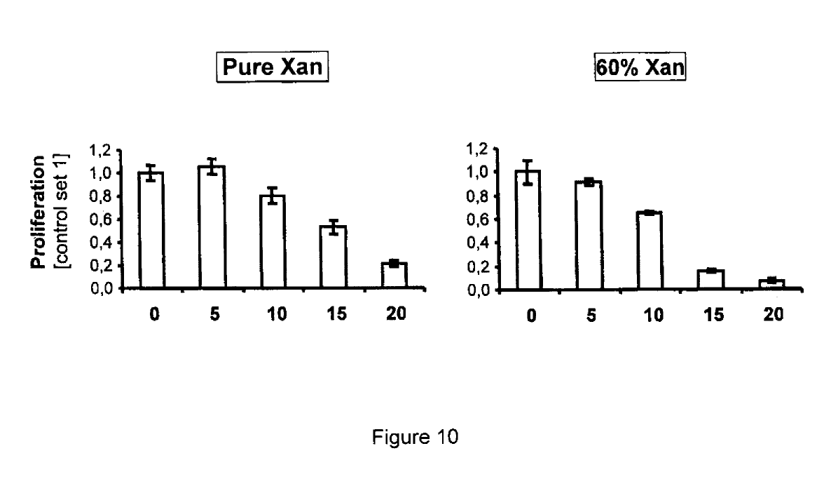

FIG. 10 shows a comparison of the lifetime (proliferation) of liver cancer cells after administration of xanthohumol in pure form (>98%) or in a form in which xanthohumol is present in a proportion of 60%. The latter case represents the xanthohumol recovered from hops extract in a conventional commercial process, containing additional natural constituents. In the figure, the lower the bars, the more cells that experience inhibition of growth.

It is demonstrated that use of 60% xanthohumol results in an even stronger effect than from xanthohumol in pure form. This is attributed to the fact that for the natural xanthohumol, the remaining constituents have a carrier function and therefore supply the active substance to the organism in a more effective manner.

1. Use of xanthohumol having the formula 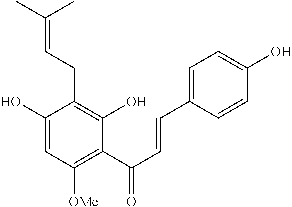
as an active substance for producing a preparation for the prevention and/or control of liver diseases.

2. Use of isoxanthohumol having the formula 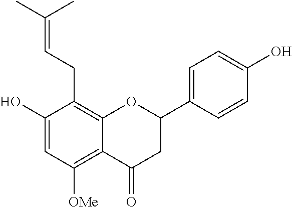
as an active substance for producing a preparation for the prevention and/or control of liver diseases.

3. Use of a metabolite of xanthohumol for the prevention and/or control of liver diseases, in particular as an active substance for producing a preparation for the prevention and/or control of liver diseases.

4. Use according to claim 3, wherein a metabolite which is produced in the liver by P450 enzymes is used as the metabolite of xanthohumol.

5. Use according to claim 3, wherein the metabolite is a glucoronide, a sulfate, a methyl [sic], or is naringenin, in particular 8-prenylnaringenin.

6. Use of a precursor which regenerates to form xanthohumol under chemical and/or physiological conditions for the prevention and/or control of liver diseases, in particular as an active substance for producing a preparation for the prevention and/or control of liver diseases.

7. Use according to claim 1, wherein the liver disease is cirrhosis of the liver or hepatic fibrosis.

8. Use according claim 1, wherein the liver disease is a viral liver inflammation, in particular hepatitis, in particular hepatitis B or hepatitis C.

9. Use according to claim 1 wherein the liver disease is nonalcoholic steatohepatitis (NASH).

10. Use according to claim 1, wherein the liver disease is liver cancer.

11. Use according to claim 1, wherein the xanthohumol or isoxanthohumol or a metabolite thereof or a precursor thereof is supplied as an active ingredient of a pharmaceutical composition together with a pharmaceutically acceptable carrier.

12. Use according to claim 1, wherein the xanthohumol or isoxanthohumol or a metabolite thereof or a precursor thereof is added as an active substance to a food.

13. Use according to claim 1, wherein the xanthohumol or isoxanthohumol or a metabolite thereof or a precursor thereof is added as an active substance to a beverage.

14. Use according to Claim l,wherein the xanthohumol or isoxanthohumol or a metabolite thereof or a precursor thereof is used to reduce or suppress the activity of free oxygen radicals.

15. Use according to claim 1, wherein the xanthohumol or isoxanthohumol or a metabolite thereof or a precursor thereof is used to influence the NF-kappa B factor, in particular to reduce or suppress the activity thereof

16. Use according to claim 1, wherein the particular active substance is used in an administration form which results in active substance concentrations of ≧5 μM, in particular ≧10 μM, in particular ≧20 μM, in particular ≧30 μM, in particular ≧40 μM, in particular ≧50 μM, in the liver.

17. Use according to claim 1, wherein the active substance is used in an administration form which results in a maximum active substance concentration of 100 μM in the liver.

18. Use according to claim 1, wherein he xanthohumol and/or isoxanthohumol is used in an administration form in such a way that the following ranges of active substance concentrations result in the liver: 1 to 100 μM, preferably 1-25 μM, preferably 1-10 μM, or 5-100 μM, preferably 10-50 μM, preferably 10-25 μM.

19. Use according to claim 1, wherein the dosage relative to the fraction of active substance is greater than 0.01 mg/kg body weight/day, preferably greater than 0.1 mg/kg body weight/day, preferably greater than 1 mg/kg body weight/day, preferably greater than 10 mg/kg body weight/day, preferably greater than 50 mg/kg body weight/day, preferably greater than 100 mg/kg body weight/day, whereby the body weight refers to the body weight of a person.

20. Use according to claim 1, wherein the dosage relative to the fraction of active substance is 161 mg/kg body weight/day, preferably less than 50 mg/kg body weight/day, preferably less than 10 mg/kg body weight/day, preferably less than 1 mg/kg body weight/day, preferably less than 0.1 mg/kg body weight/ day, whereby the body weight refers to the body weight of a person.

21. Use according to claim 1, wherein the dosage relative to the fraction of active substance is in a range of 0.01 to 161 mg/kg body weight/day, preferably 0.05 to 120 mg/kg body weight/day, preferably 0.1 to 100 mg/kg body weight/day, preferably 0.5 to 80 mg/kg body weight/day, preferably 1 to 80 mg/kg body weight/day, preferably 5 to 80 mg/kg body weight/day, preferably 10 to 80 mg/kg body weight/day, whereby the body weight refers to the body weight of a person.

22. Use according to claim 1, wherein a composition is used as active substance in which the xanthohumol or isoxanthohumol is used in a proportion in a range of 0.1 wt-%-99 wt-%, preferably 5 wt-%-99 wt-%, preferably 10 wt-%-99 wt-%, preferably 20 wt-%-99 wt-%, preferably 30 wt-%-99 wt-%, preferably 40 wt-%-99 wt-%, preferably 50 wt-%-99 wt-%, preferably 60 wt-%-99 wt-%, preferably 70 wt-%-99 wt-%.

23. Use according to claim 1, wherein the xanthohumol or isoxanthohumol is used in pure form.

24. Use according to claim 1, wherein the xanthohumol or isoxanthohumol is used in synthesized form.

25. Use according to claim 1, wherein the xanthohumol and/or
the isoxanthohumol and/or
a metabolite thereof and/or
a precursor thereof
are used in combination with at least one additional active substance.

26. Use according to claim 1, wherein the xanthohumol or isoxanthohumol or a metabolite thereof or a precursor thereof is used in combination with or on the basis of a salt, in particular an alkali or alkaline earth salt.

27. Use according to claim 1, wherein the xanthohumol or isoxanthohumol or a metabolite thereof or a precursor thereof is used in the form of a liquid, suspension, or emulsion, in the form of nanoparticles, or as a powder or gel.

28. Use according to claim 1, wherein xanthohumol and/or isoxanthohumol and/or a metabolite thereof or a precursor thereof is added to appropriately control the activity mechanism of liver inflammation, the activity mechanism of cirrhosis of the liver or hepatic fibrosis, and the activity mechanism of development of liver cancer and the activity mechanism of progression of liver cancer.

29. Use of xanthohumol having the formula 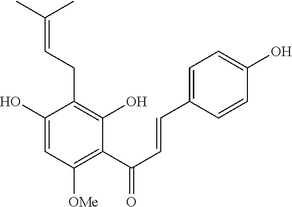
as an active substance for producing a food supplement for regular prophylactic intake by persons with adiposis and/or diabetes for the prevention of liver diseases caused by adiposis and/or diabetes.

30. Use according to claim 2, wherein the liver disease is cirrhosis of the liver or hepatic fibrosis.

31. Use according to claim 3, wherein the liver disease is cirrhosis of the liver or hepatic fibrosis.

32. Use according to claim 6, wherein the liver disease is cirrhosis of the liver or hepatic fibrosis.

33. Use according claim 2, wherein the liver disease is a viral liver inflammation, in particular hepatitis, in particular hepatitis B or hepatitis C.

34. Use according claim 3, wherein the liver disease is a viral liver inflammation, in particular hepatitis, in particular hepatitis B or hepatitis C.

35. Use according claim 6, wherein the liver disease is a viral liver inflammation, in particular hepatitis, in particular hepatitis B or hepatitis C.

36. Use according to claim 2, wherein the liver disease is nonalcoholic steatohepatitis (NASH).

37. Use according to claim 3, wherein the liver disease is nonalcoholic steatohepatitis (NASH).

38. Use according to claim 6, wherein the liver disease is nonalcoholic steatohepatitis (NASH).

39. Use according to claim 2, wherein the liver disease is liver cancer.

40. Use according to claim 3, wherein the liver disease is liver cancer.

41. Use according to claim 6, wherein the liver disease is liver cancer.

42. Use according to claim 2, wherein the xanthohumol or isoxanthohumol or a metabolite thereof or a precursor thereof is supplied as an active ingredient of a pharmaceutical composition together with a pharmaceutically acceptable carrier.

43. Use according to claim 3, wherein the xanthohumol or isoxanthohumol or a metabolite thereof or a precursor thereof is supplied as an active ingredient of a pharmaceutical composition together with a pharmaceutically acceptable carrier.

44. Use according to claim 6, wherein the xanthohumol or isoxanthohumol or a metabolite thereof or a precursor thereof is supplied as an active ingredient of a pharmaceutical composition together with a pharmaceutically acceptable carrier.

45. Use according to claim 2, wherein the xanthohumol or isoxanthohumol or a metabolite thereof or a precursor thereof is added as an active substance to a food.

46. Use according to claim 3, wherein the xanthohumol or isoxanthohumol or a metabolite thereof or a precursor thereof is added as an active substance to a food.

47. Use according to claim 6, wherein the xanthohumol or isoxanthohumol or a metabolite thereof or a precursor thereof is added as an active substance to a food.

48. Use according to claim 2, wherein the xanthohumol or isoxanthohumol or a metabolite thereof or a precursor thereof is added as an active substance to a beverage.

49. Use according to claim 3, wherein the xanthohumol or isoxanthohumol or a metabolite thereof or a precursor thereof is added as an active substance to a beverage.

50. Use according to claim 6, wherein the xanthohumol or isoxanthohumol or a metabolite thereof or a precursor thereof is added as an active substance to a beverage.

51. Use according to claim 2,wherein the xanthohumol or isoxanthohumol or a metabolite thereof or a precursor thereof is used to reduce or suppress the activity of free oxygen radicals.

52. Use according to claim 3,wherein the xanthohumol or isoxanthohumol or a metabolite thereof or a precursor thereof is used to reduce or suppress the activity of free oxygen radicals.

53. Use according to claim 6,wherein the xanthohumol or isoxanthohumol or a metabolite thereof or a precursor thereof is used to reduce or suppress the activity of free oxygen radicals.

54. Use according to claim 2, wherein the xanthohumol or isoxanthohumol or a metabolite thereof or a precursor thereof is used to influence the NF-kappa B factor, in particular to reduce or suppress the activity thereof.

55. Use according to claim 3, wherein the xanthohumol or isoxanthohumol or a metabolite thereof or a precursor thereof is used to influence the NF-kappa B factor, in particular to reduce or suppress the activity thereof.

56. Use according to claim 6, wherein the xanthohumol or isoxanthohumol or a metabolite thereof or a precursor thereof is used to influence the NF-kappa B factor, in particular to reduce or suppress the activity thereof.

57. Use according to claim 2, wherein a composition is used as active substance in which the xanthohumol or isoxanthohumol is used in a proportion in a range of 0.1 wt-%-99 wt-%, preferably 5 wt-%-99 wt-%, preferably 10 wt-%-99 wt-%, preferably 20 wt-%-99 wt-%, preferably 30 wt-%-99 wt-%, preferably 40 wt-%-99 wt-%, preferably 50 wt-%-99 wt-%, preferably 60 wt-%-99 wt-%, preferably 70 wt-%-99 wt-%.

58. Use according to claim 2, whereinxanthohumol and/or isoxanthohumol and/or a metabolite thereof or a precursor thereof is added to appropriately control the activity mechanism of liver inflammation, the activity mechanism of cirrhosis of the liver or hepatic fibrosis, and the activity mechanism of development of liver cancer and the activity mechanism of progression of liver cancer.

59. Use according to claim 3, whereinxanthohumol and/or isoxanthohumol and/or a metabolite thereof or a precursor thereof is added to appropriately control the activity mechanism of liver inflammation, the activity mechanism of cirrhosis of the liver or hepatic fibrosis, and the activity mechanism of development of liver cancer and the activity mechanism of progression of liver cancer.

60. Use according to claim 6, whereinxanthohumol and/or isoxanthohumol and/or a metabolite thereof or a precursor thereof is added to appropriately control the activity mechanism of liver inflammation, the activity mechanism of cirrhosis of the liver or hepatic fibrosis, and the activity mechanism of development of liver cancer and the activity mechanism of progression of liver cancer.
